(19) United States
(12) Patent Application Publication
(10) Pub. No.: US 2008/0107692 A1
(43) Pub. Date: May 8, 2008

(54) COMPOSITIONS AND METHODS FOR THE DETECTION OF TRYPANOSOMA CRUZI INFECTION

(75) Inventor: Steven Reed, Bellevue, WA (US)

Correspondence Address: SEED INTELLECTUAL PROPERTY LAW GROUP PLLC, 701 FIFTH AVE, SUITE 5400, SEATTLE, WA, 98104, US

(73) Assignee: INFECTIOUS DISEASE RESEARCH INSTITUTE, Seattle, WA (US)

(21) Appl. No.: 11/743,534

(22) Filed: May 2, 2007

Related U.S. Application Data
(63) Continuation-in-part of application No. PCT/US06/42907, filed on Nov. 2, 2006.

(51) Int. Cl. A61K 39/005; A61P 33/02; C12N 1/21; C12N 15/12; C12N 15/63; C12N 5/10; G01N 33/53

(52) U.S. Cl. 424/269.1; 435/252.3; 435/252.33; 435/320.1; 435/325; 435/348; 435/358; 435/7.2; 530/350; 536/23.7

(57) ABSTRACT

The invention provides antigenic T. cruzi polypeptides, polynucleotides, and fusions thereof, as well as compositions comprising same. Also provided are diagnostic kits and methods for the diagnosis of T. cruzi infection and in screening blood supplies.

### CROSS-REFERENCE TO RELATED APPLICATION

This application is a continuation-in-part of International Patent Application No. PCT/US06/42907 filed Nov. 2, 2006, now pending, which application is incorporated herein by reference in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 480239_{—}405C1_SEQUENCE_LISTING.txt. The text file is 11 KB, was created on May 2, 2007, and is being submitted electronically via EFS-Web, concurrent with the filing of the specification.

### FIELD OF THE INVENTION

The present invention relates generally to the diagnosis of Trypanosoma cruzi (T. cruzi) infection. More specifically, the invention relates to the use of T. cruzi antigenic polypeptides and fusion polypeptides in methods for screening individuals and blood supplies for T. cruzi infection.

### BACKGROUND OF THE INVENTION

Protozoan parasites are a serious health threat in many areas of the world. Trypanosoma cruzi (T. cruzi) is one such parasite that infects millions of individuals. Ten to thirty percent of individuals infected with T. cruzi develop chronic symptomatic Chagas' disease, which may in turn lead to heart disease and a variety of immune system disorders. T. cruzi infection has long been a public health problem in Central and South America. It is estimated that 18 million people worldwide are chronically infected with T. cruzi, but available drug treatments lack efficacy and often cause serious side effects.

The most significant route of transmission in areas where the disease is endemic is through contact with an infected triatomid insect. However in other areas blood transfusions are the dominant means of transmission. Accordingly, in order to inhibit the transmission of T. cruzi, it is necessary to develop accurate methods for both diagnosing T. cruzi infection in individuals and for screening blood supplies. Blood bank screening is particularly important in South America, where 0.1%-62% of blood samples may be infected and where the parasite is frequently transmitted by blood transfusion. Due to high flow of immigrants to the US from many Central and South American countries where T. cruzi infection is endemic, the US blood supply is becoming at high risk for contamination from T. cruzi infected blood donors. While there are a limited number of tests available for diagnosing infection in individuals, there is currently no FDA approved test available in the US for blood donor screening for T. cruzi infection.

The diagnosis of T. cruzi infection has been problematic because accurate methods for detecting the parasite that are suitable for routine use have been unavailable. During the acute phase of infection, which may last for decades, the infection may remain quiescent and the host may be asymptomatic. As a result, serological tests for T. cruzi infection are the most reliable and the most commonly used form of diagnosis. Such diagnoses are complicated, however, by the complex life cycle of the parasite and the diverse immune responses of the host. The parasite passes through an epimastigote stage in the insect vector and two main stages in the mammalian host. One host stage is present in blood (the trypomastigote stage), while a second stage is intracellular (the amastigote stage). The multiple stages result in a diversity of antigens being presented by the parasite during infection. In addition, immune responses to protozoan infection are complex, involving both humoral and cell-mediated responses to the array of parasite antigens.

While detection of antibodies against parasite antigens is the most common and reliable method of diagnosing clinical and subclinical infections, current tests for T. cruzi infection are generally insensitive, lack specificity, and are not suitable for screening of blood supplies. Most serological tests use whole or lysed T. cruzi and require positive results on two of three tests, including complement fixation, indirect immunofluorescence, passive agglutination or ELISA, to accurately detect T. cruzi infection. The cost and difficulty of such tests has hindered the widespread screening of blood or sera in many endemic areas.

U.S. Pat. Nos. 5,876,734 and 6,228,601 disclose compositions useful for diagnosing Chagas' disease that comprise a non-repetitive region of the T. cruzi protein TCR27, and fusion polypeptides including such regions. U.S. Pat. No. 6,419,933 discloses a fusion polypeptide referred to as TcF that contains the four antigenic T. cruzi peptides PEP-2, TcD, TcE and TcLo1.2, together with methods for the use of the fusion polypeptide in the detection of T. cruzi infection. While TcF is highly reactive with T. cruzi-infected sera from South America, it exhibits low activity and is occasionally negative with Central American sera. U.S. Pat. No. 6,458,922 discloses an assay for T. cruzi infection that employs compositions comprising at least six antigenic T. cruzi peptides selected from the group consisting of: SAPA, CRA, FRA, TcD, Tc24, Ag39 and MAP. Published US Patent Application No. US-2004/0132077-A1 discloses recombinant polypeptides and fusion polypeptides (referred to as FP3, FP4, FP5, FP6, FP7, FP8, FP9 and FP10) for diagnosing T. cruzi infection. The disclosed fusion polypeptides comprise modified versions of previously identified T. cruzi epitopes, including TCR27, TCR39, SAPA and MAP.

While various approaches have been described for the diagnosis of T. cruzi infection, there remains a need for improved strategies that are reliable, sensitive and amenable to large-scale use in all geographic areas for screening individuals and blood supplies. The present invention addresses these needs and offers other related advantages.

### SUMMARY OF THE INVENTION

The present invention provides isolated T. cruzi polypeptides, fusion polypeptides, and compositions thereof, as well as methods for detecting T. cruzi infection in individuals and in biological samples, including blood supplies. The polypeptides and compositions may be employed to detect and/or screen for T. cruzi infection in essentially all geographical areas where Chagas' disease is present and with improved sensitivity compared to assays currently in use.

According to one aspect of the invention, there are provided isolated polypeptides comprising an amino acid sequence selected from the group consisting of Tc5 (SEQ ID NO: 2), Tc48 (SEQ ID NO: 5), Tc60 (SEQ ID NO: 7) and Tc70 (SEQ ID NO: 9), or a variant thereof having at least 90% identity thereto. In a related aspect, the invention provides isolated polypeptides comprising at least an immunogenic portion of an amino acid sequence selected from the group consisting of Tc5 (SEQ ID NO: 2), Tc48 (SEQ ID NO: 5), Tc60 (SEQ ID NO: 7) and Tc70 (SEQ ID NO: 9).

Also provided by the present invention are fusion polypeptides comprising at least one isolated polypeptide of the invention in combination with one or more fusion partners.

Polynucleotides encoding the inventive polypeptides and fusion polypeptides, expression vectors comprising such polynucleotides, and host cells transformed or transfected with such expression vectors, are also provided by the present invention.

In another aspect of the invention, methods are provided for detecting T. cruzi infection in a biological sample, comprising: (a) contacting the biological sample with a polypeptide of the invention; and (b) detecting in the biological sample the presence of antibodies that bind to the polypeptide, and thereby detecting T. cruzi infection in the biological sample. In a particular embodiment, the biological sample tested in the method is a biological sample selected from the group consisting of blood, serum, plasma, saliva, cerebrospinal fluid and urine.

In yet another aspect, the invention provides diagnostic kits for detecting T. cruzi infection in a biological sample, comprising a polypeptide of the invention and a detection reagent. In a particular embodiment, the detection reagent comprises a reporter group. In a more particular embodiment, the reporter group is selected from the group consisting of enzymes, substrates, cofactors, inhibitors, dyes, radionuclides, luminescent groups, fluorescent groups and biotin.

According to another aspect of the invention compositions are provided comprising a polypeptide or fusion polypeptide of the invention in combination with a physiologically acceptable carrier and/or immunostimulant.

In yet another aspect, the invention provides a method for the prevention or treatment of T. cruzi infection comprising administering to a patient a composition of the invention. In a related aspect, methods are provided for inducing protective immunity against Chagas' disease in a patient by administering a composition of the invention.

The above-mentioned and additional features of the present invention and the manner of obtaining them will become apparent, and the invention will be best understood by reference to the following more detailed description. All references disclosed herein are hereby incorporated by reference in their entirety as if each was incorporated individually.

### BRIEF DESCRIPTION OF THE FIGURES

FIGS. 1A and 1B show the isolated DNA sequence and corresponding amino acid sequence, respectively, for TC5. The insert is shown in bold font, and the flanking sequence in non-bold font.

FIGS. 2A and 2B show the isolated DNA sequence and corresponding amino acid sequence, respectively, for TC48. The insert is shown in bold font, and the flanking sequence in non-bold font.

FIGS. 3A and 3B show the isolated DNA sequence and corresponding amino acid sequence, respectively, for TC60. The insert is shown in bold font, and the flanking sequence in non-bold font.

FIGS. 4A and 4B show the isolated DNA sequence and corresponding amino acid sequence, respectively, for TC70. The insert is shown in bold font, and the flanking sequence in non-bold font.

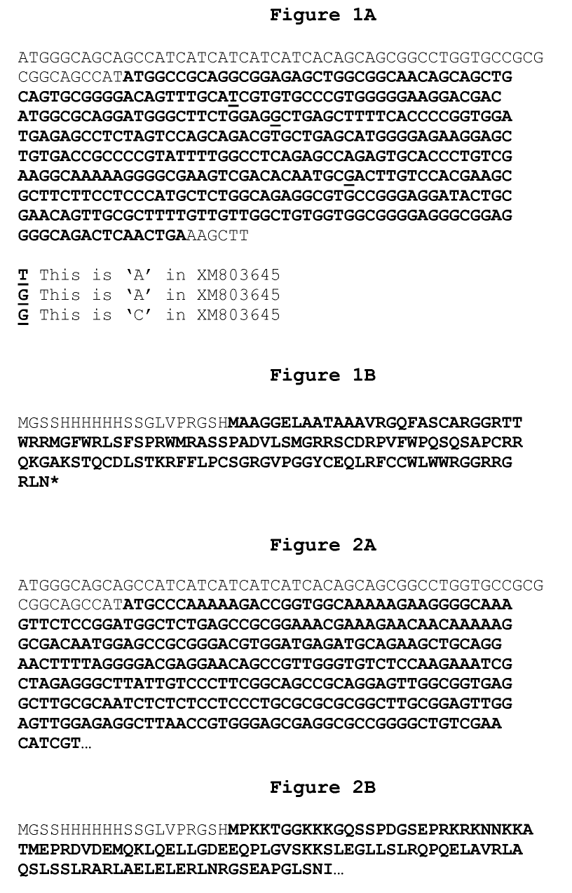

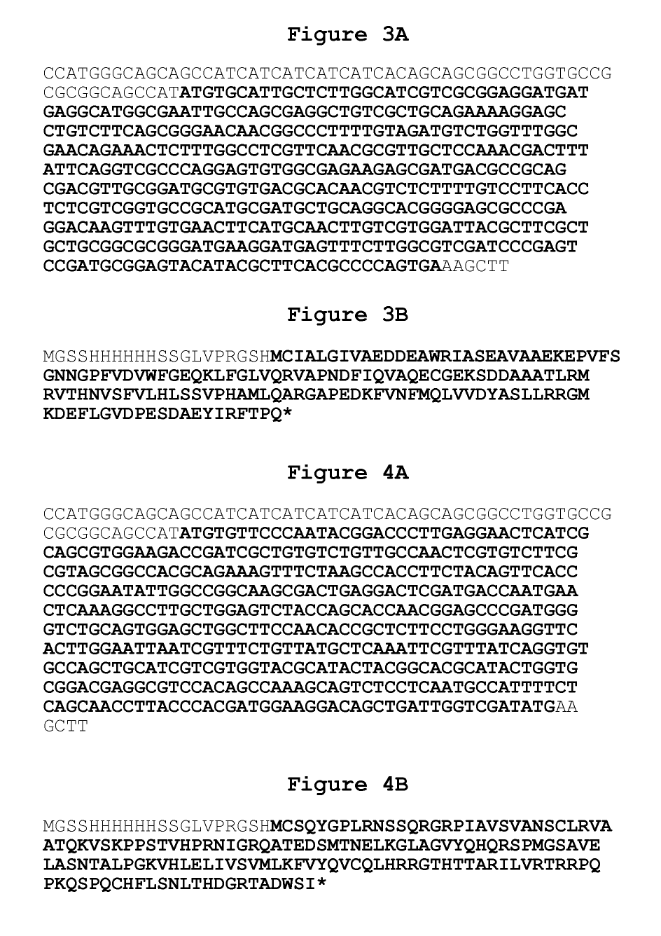

### BRIEF DESCRIPTION OF SEQUENCE IDENTIFIERS

SEQ ID NO: 1 is a DNA sequence for the identified T. cruzi antigen referred to as Tc5.

SEQ ID NO: 2 is an amino acid sequence for the identified T. cruzi antigen referred to as Tc5.

SEQ ID NO: 3 is a DNA sequence for the identified T. cruzi antigen referred to as Tc48.

SEQ ID NO: 4 is an identified partial amino acid sequence for the identified T. cruzi antigen referred to as Tc48.

SEQ ID NO: 5 is a full length amino acid sequence for the identified T. cruzi antigen referred to as Tc48.

SEQ ID NO: 6 is a DNA sequence for the identified T. cruzi antigen referred to as Tc60.

SEQ ID NO: 7 is an amino acid sequence for the identified T. cruzi antigen referred to as Tc60.

SEQ ID NO: 8 is a DNA sequence for the identified T. cruzi antigen referred to as Tc70.

SEQ ID NO: 9 is an amino acid sequence for the identified T. cruzi antigen referred to as Tc70.

### DETAILED DESCRIPTION OF THE INVENTION

As noted above, the present invention is generally directed to compositions and methods for detecting T. cruzi infection in individuals and for screening blood supplies for T. cruzi infection. The compositions of this invention generally comprise a T. cruzi polypeptide (including immunogenic portions, epitopes and/or variants thereof), as described herein, or a polynucleotide encoding such polypeptides, wherein the T. cruzi polypeptide is selected from the group consisting of Tc5, Tc48, Tc60 and/or Tc70. Fusion polypeptides comprising one or more T. cruzi polypeptides (including immunogenic portions, epitopes and/or variants thereof), as described herein, are also provided, as are polynucleotides encoding such fusion polypeptides.

As used herein, the term “polypeptide” encompasses amino acid chains of any length, including full-length proteins, wherein amino acid residues are linked by covalent peptide bonds. Polypeptides disclosed herein may be naturally purified products, or may be produced partially or wholly using recombinant techniques. Such polypeptides may be glycosylated with mammalian or other eukaryotic carbohydrates or may be non-glycosylated. A polypeptide comprising an epitope or immunogenic portion may consist entirely of the epitope or portion, or may contain additional sequences. The additional sequences may be derived from the native antigen or may be heterologous, and such sequences may (but need not) be antigenic.

Generally, a polypeptide of the invention will be an isolated polypeptide and may be a fragment (e.g., an antigenic/immunogenic portion) from an amino acid sequence disclosed herein, or may comprise an entire amino acid sequence disclosed herein. Polypeptides of the invention, antigenic/immunogenic fragments thereof, and other variants may be prepared using conventional recombinant and/or synthetic techniques.

In certain embodiments, the polypeptides of the invention are antigenic/immunogenic, i.e., they react detectably within an immunoassay (such as an ELISA or T cell stimulation assay) with antisera and/or T cells from an infected subject. Screening for immunogenic activity can be performed using techniques well known to the skilled artisan. For example, such screens can be performed using methods such as those described in Harlow and Lane, Antibodies: A Laboratory Manual, Cold Spring Harbor Laboratory, 1988. In one illustrative example, a polypeptide may be immobilized on a solid support and contacted with patient sera to allow binding of antibodies within the sera to the immobilized polypeptide. Unbound sera may then be removed and bound antibodies detected using, for example, ¹²⁵I-labeled Protein A.

As would be recognized by the skilled artisan, polypeptides of the invention include immunogenic portions/fragments of the polypeptides disclosed herein. An “immunogenic portion,” as used herein, is a fragment of an immunogenic polypeptide of the invention that itself is immunologically reactive (i.e., specifically binds) with the B-cells and/or T cell surface antigen receptors that recognize the polypeptide. Immunogenic portions may generally be identified using well known techniques, such as those summarized in Paul, Fundamental Immunology, 3rd ed., 243-247 (Raven Press, 1993) and references cited therein. Such techniques include screening polypeptides for the ability to react with antigen-specific antibodies, antisera and/or T cell lines or clones. As used herein, antisera and antibodies are “antigen-specific” if they specifically bind to an antigen (i.e., they react with the protein in an immunoassay, and do not react detectably with unrelated proteins). Such antisera and antibodies may be prepared as described herein, and using well-known techniques.

In a particular embodiment, an antigenic/immunogenic portion is a portion that reacts with a biological sample (e.g, sera from a T. cruzi infected patient or sample) at a level that is not substantially less than the reactivity of the full-length polypeptide (e.g., in an ELISA and/or T cell reactivity assay). In one embodiment, the level of immunogenic activity/reactivity of the immunogenic portion is at least about 50%, preferably at least about 70% and most preferably greater than about 90% of the immunogenicity for the full-length polypeptide. In another embodiment, an immunogenic portion has a level of immunogenic activity/reactivity greater than that of the corresponding full-length polypeptide, e.g., having greater than about 100% or 150% or more immunogenic activity.

A polypeptide composition of the invention may also comprise one or more polypeptides that are immunologically reactive with T cells and/or antibodies generated against a polypeptide of the invention, particularly a polypeptide having an amino acid sequence disclosed herein, or to an immunogenic fragment or variant thereof.

In another embodiment of the invention, polypeptides are provided that comprise one or more polypeptides that are capable of eliciting T cells and/or antibodies that are immunologically reactive with one or more polypeptides described herein, or one or more polypeptides encoded by contiguous polynucleotide sequences contained in the polynucleotide sequences disclosed herein, or immunogenic fragments or variants thereof, or to one or more polynucleotide sequences which hybridize to one or more of these sequences under conditions of moderate to high stringency.

The present invention also provides polypeptide portions/fragments, including antigenic/immunogenic portions/fragments, comprising at least about 5, 10, 15, 20, 25, 50, or 100 contiguous amino acids, or more, including all intermediate lengths, of a polypeptide composition set forth herein, or those encoded by a polynucleotide sequence set forth herein.

The terms “fusion polypeptide” and “fusion protein” are used interchangeably and include polypeptides in which one or more of the T. cruzi polypeptides described herein (including immunogenic portions, epitopes and/or variants thereof) is fused with another T. cruzi polypeptide (or immunogenic portion, epitope and/or variant thereof) and/or with another heterologous sequence that is not a T. cruzi polypeptide. Also provided by the invention are polynucleotides encoding fusion polypeptides/proteins.

The fusion components that make up a fusion polypeptide are generally covalently linked, either directly or via an amino acid linker. For example, the polypeptides forming the fusion polypeptide are typically linked C-terminus to N-terminus, although they can also be linked C-terminus to C-terminus, N-terminus to N-terminus, or N-terminus to C-terminus. The polypeptide components of the fusion polypeptide can be in any order. Fusion polypeptides can also include conservatively modified variants, polymorphic variants, alleles, mutants, subsequences, interspecies homologs, and immunogenic fragments of the antigens that make up the fusion polypeptide.

Thus, the fusion polypeptides of the invention generally comprise at least one antigenic polypeptide as described herein, and further comprise other unrelated sequences, such as another T. cruzi antigen sequence or a sequence that assists in providing T helper epitopes (an immunological fusion partner), preferably T helper epitopes recognized by humans, or that assists in expressing the protein (an expression enhancer) at higher yields than the native recombinant protein. Certain preferred illustrative partners are both immunological and expression-enhancing fusion partners. Other fusion partners may be selected so as to increase the solubility of the protein or to enable the protein to be targeted to desired intracellular compartments. Still further fusion partners include affinity tags, which facilitate purification of the protein.

In a particular embodiment, epitopes of different antigens, or variants thereof, are joined, for example through a peptide linkage, into a single amino acid chain. The amino acid chain thus formed may be either linear or branched. The epitopes may be joined directly (i.e., with no intervening amino acids) or may be joined by way of a linker sequence that does not significantly alter the antigenic properties of the epitopes. The peptide epitopes may also be linked through non-peptide linkages, such as hetero- or homo-bifunctional agents that chemically or photochemically couple between specific functional groups on the peptide epitopes such as through amino, carboxyl, or sulfhydryl groups. Bifunctional agents which may be usefully employed in the combination polypeptides of the present invention are well known to those of skill in the art. Epitopes may also be linked by means of a complementary ligand/anti-ligand pair, such as avidin/biotin, with one or more epitopes being linked to a first member of the ligand/anti-ligand pair and then being bound to the complementary member of the ligand/anti-ligand pair either in solution or in solid phase. A fusion polypeptide may contain epitopes of one or more other T. cruzi antigens, linked to an epitope described herein.

A polynucleotide encoding a fusion protein of the present invention is constructed using known recombinant DNA techniques to assemble separate polynucleotides encoding the first and second polypeptides into an appropriate expression vector. The 3′ end of a polynucleotide encoding a first polypeptide is ligated, with or without a peptide linker, to the 5′ end of a polynucleotide encoding a second polypeptide so that the reading frames of the sequences are in phase to permit mRNA translation of the two polynucleotides into a single fusion protein that retains the biological activity of both the first and the second polypeptides.

As noted above, a peptide linker sequence may be employed to separate the first and the second polypeptides by a distance sufficient to ensure that each polypeptide folds into its secondary and tertiary structures. Such a peptide linker sequence is incorporated into the fusion protein using standard techniques well known in the art. Suitable peptide linker sequences may be chosen, for example, based on one or more of the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40:39-46, 1985; Murphy et al., Proc. Natl. Acad. Sci. USA 83:8258-8262, 1986; U.S. Pat. No. 4,935,233 and U.S. Pat. No. 4,751,180. The linker sequence may be from 1 to about 50 amino acids in length. Peptide linker sequences are not required when the first and second polypeptides have non-essential N-terminal amino acid regions that can be used to separate the functional domains and prevent steric interference. The ligated polynucleotides encoding the fusion proteins are cloned into suitable expression systems using techniques known to those of ordinary skill in the art.

The present invention further provides polynucleotides that encode a polypeptide or fusion polypeptide of the present invention. Polynucleotides that comprise complements of such polynucleotide sequences, reverse complements of such polynucleotide sequences, or reverse sequences of such polynucleotide sequences, together with variants of such sequences, are also provided.

The terms “complement(s),” “reverse complement(s),” and “reverse sequence(s),” as used herein, can be understood by reference to the following example. For the sequence 5′ AGGACC 3′, the complement, reverse complement, and reverse sequence are as follows:

| | |
|---|---|
| complement | 3′ TCCTGG 5′ |
| reverse complement | 3′ GGTCCT 5′ |
| reverse sequence | 5′ CCAGGA 3′. |

In certain embodiments, sequences that are complements of a specifically recited polynucleotide sequence are complementary over the entire length of the specific polynucleotide sequence.

The term “polynucleotide(s),” as used herein, means a single or double-stranded polymer of deoxyribonucleotide or ribonucleotide bases and includes DNA and corresponding RNA molecules, including HnRNA and mRNA molecules, both sense and anti-sense strands, and comprehends cDNA, genomic DNA and recombinant DNA, as well as wholly or partially synthesized polynucleotides. An HnRNA molecule contains introns and corresponds to a DNA molecule in a generally one-to-one manner. An mRNA molecule corresponds to an HnRNA and DNA molecule from which the introns have been excised. A polynucleotide may consist of an entire gene, or any portion thereof. Operable anti-sense polynucleotides may comprise a fragment of the corresponding polynucleotide, and the definition of “polynucleotide” therefore includes all such operable anti-sense fragments.

All of the polypeptides, fusion polypeptides and polynucleotides described herein are isolated and purified, as those terms are commonly used in the art. Preferably, the polypeptides, fusion polypeptides and polynucleotides are at least about 80% pure, more preferably at least about 90% pure, and most preferably at least about 99% pure.

The compositions and methods of the present invention also encompass variants of the above polypeptides, fusion polypeptides and polynucleotides. As used herein, the term “variant” includes nucleotide or amino acid sequences different from the specifically identified sequences, wherein one or more nucleotides or amino acid residues is deleted, substituted, or added. Variants may be naturally occurring allelic variants, or non-naturally occurring variants. Variant sequences (polynucleotide or polypeptide) preferably exhibit at least 75%, more preferably at least 80%, more preferably at least 85%, more preferably at least 90%, more preferably yet at least 95%, and most preferably, at least 98% identity to a sequence of the present invention. The percentage identity is determined by aligning the two sequences to be compared as described below, determining the number of identical residues in the aligned portion, dividing that number by the total number of residues in the inventive (queried) sequence, and multiplying the result by 100.

In addition to exhibiting the recited level of sequence identity, variant sequences of the present invention preferably exhibit a functionality that is substantially similar to the functionality of the specific sequences disclosed herein. Variant fusion polypeptide sequences thus preferably retain the antigenic and diagnostic properties of the fusion polypeptides disclosed herein. Preferably a variant polypeptide or fusion polypeptide sequence will generate at least 80%, more preferably at least 85%, more preferably at least 90%, more preferably yet at least 95% and most preferably 100% of the response generated by the specifically identified polypeptide or fusion polypeptide sequence in an antibody binding assay, such as an ELISA assay. Such variants may generally be identified by modifying one of the polypeptide or fusion polypeptide sequences disclosed herein, and evaluating the antigenic and/or diagnostic properties of the modified polypeptide or fusion polypeptide using, for example, the representative procedures described herein. Suitable assays for evaluating reactivity with T. cruzi-infected sera, such as an enzyme linked immunosorbent assay (ELISA), are described in more detail below, and in Harlow and Lane, Antibodies: A Laboratory Manual, Cold Spring Harbor Laboratory, 1988.

Variant sequences often differ from the specifically identified sequence only by conservative substitutions, deletions or modifications. As used herein, a “conservative substitution” is one in which an amino acid is substituted for another amino acid that has similar properties, such that one skilled in the art of peptide chemistry would expect the secondary structure and hydropathic nature of the polypeptide to be substantially unchanged. In general, the following groups of amino acids represent conservative changes: (1) ala, pro, gly, glu, asp, gin, asn, ser, thr; (2) cys, ser, tyr, thr; (3) val, ile, leu, met, ala, phe; (4) lys, arg, his; and (5) phe, tyr, trp, his. Variants may also, or alternatively, contain other modifications, including the deletion or addition of amino acids that have minimal influence on the antigenic properties, secondary structure and hydropathic nature of the polypeptide. For example, a polypeptide may be conjugated to a signal (or leader) sequence at the N-terminal end of the protein which co-translationally or post-translationally directs transfer of the protein. The polypeptide may also be conjugated to a linker or other sequence for ease of synthesis, purification or identification of the polypeptide (e.g., poly-His), or to enhance binding of the polypeptide to a solid support. For example, a polypeptide may be conjugated to an immunoglobulin Fc region.

Polypeptide and polynucleotide sequences may be aligned, and percentages of identical nucleotides in a specified region may be determined against another polynucleotide, using computer algorithms that are publicly available. Two exemplary algorithms for aligning and identifying the identity of polynucleotide sequences are the BLASTN and FASTA algorithms. The alignment and identity of polypeptide sequences may be examined using the BLASTP and algorithm. BLASTX and FASTX algorithms compare nucleotide query sequences translated in all reading frames against polypeptide sequences. The FASTA and FASTX algorithms are described in Pearson and Lipman, Proc. Natl. Acad. Sci. USA 85:2444-2448, 1988; and in Pearson, Methods in Enzymol. 183:63-98, 1990. The FASTA software package is available from the University of Virginia, Charlottesville, Va. 22906-9025. The FASTA algorithm, set to the default parameters described in the documentation and distributed with the algorithm, may be used in the determination of polynucleotide variants. The readme files for FASTA and FASTX Version 2.0× that are distributed with the algorithms describe the use of the algorithms and describe the default parameters.

The BLASTN software is available on the NCBI anonymous FTP server and is available from the National Center for Biotechnology Information (NCBI), National Library of Medicine, Building 38A, Room 8N805, Bethesda, Md. 20894. The BLASTN algorithm Version 2.0.6 [Sep. 10, 1998] and Version 2.0.11 [Jan. 20, 2000] set to the default parameters described in the documentation and distributed with the algorithm, is preferred for use in the determination of variants according to the present invention. The use of the BLAST family of algorithms, including BLASTN, is described at NCBI's website and in the publication of Altschul, et al., “Gapped BLAST and PSI-BLAST: a new generation of protein database search programs,” Nucleic Acids Res. 25:3389-3402, 1997.

The “hits” to one or more database sequences by a queried sequence produced by BLASTN, BLASTP, FASTA, or a similar algorithm, align and identify similar portions of sequences. The hits are arranged in order of the degree of similarity and the length of sequence overlap. Hits to a database sequence generally represent an overlap over only a fraction of the sequence length of the queried sequence.

The percentage identity of a polynucleotide or polypeptide sequence is determined by aligning polynucleotide and polypeptide sequences using appropriate algorithms, such as BLASTN or BLASTP, respectively, set to default parameters; identifying the number of identical nucleic or amino acids over the aligned portions; dividing the number of identical nucleic or amino acids by the total number of nucleic or amino acids of the polynucleotide or polypeptide of the present invention; and then multiplying by 100 to determine the percentage identity.

In general, T. cruzi polypeptides and fusion polypeptides, and polynucleotide sequences encoding such polypeptides and fusion polypeptides, may be prepared using any of a variety of procedures. For example, a T. cruzi cDNA or genomic DNA expression library may be screened with pools of sera from T. cruzi-infected individuals. Such screens may generally be performed using techniques well known to those of ordinary skill in the art, such as those described in Sambrook et al., Molecular Cloning: A Laboratory Manual, Cold Spring Harbor Laboratories, Cold Spring Harbor, N.Y., 1989. Briefly, the bacteriophage library may be plated and transferred to filters. The filters may then be incubated with serum and a detection reagent. In the context of this invention, a “detection reagent” is any compound capable of binding to the antibody-antigen complex, which may then be detected by any of a variety of means known to those of ordinary skill in the art. Typical detection reagents for screening purposes contain a “binding agent,” such as Protein A, Protein G, IgG or a lectin, coupled to a reporter group. Illustrative reporter groups include, but are not limited to, enzymes, substrates, cofactors, inhibitors, dyes, radionuclides, luminescent groups, fluorescent groups and biotin. In a more particular example, the reporter group is horseradish peroxidase (HRP), which may be detected by incubation with a substrate such as tetramethylbenzidine (TMB) or 2,2′-azino-di-3-ethylbenzthiazoline sulfonic acid. Plaques containing cDNAs that express a protein that binds to an antibody in the serum may be isolated and purified by techniques known to those of ordinary skill in the art. Appropriate methods may be found, for example, in Sambrook et al., Molecular Cloning: A Laboratory Manual, Cold Spring Harbor Laboratories, Cold Spring Harbor, N.Y., 1989.

Alternatively, polynucleotides encoding the polypeptides and fusion polypeptides disclosed herein may be amplified from T. cruzi genomic DNA or cDNA via polymerase chain reaction (PCR). For this approach, sequence-specific primers may be designed based on the polynucleotide sequence, and may be purchased or synthesized. An amplified portion of the DNA sequences may then be used to isolate the full length genomic or cDNA clones using well known techniques, such as those described in Sambrook et al., Molecular Cloning: A Laboratory Manual, Cold Spring Harbor Laboratories, Cold Spring Harbor, N.Y. (1989).

Polypeptides having fewer than about 100 amino acids, and generally fewer than about 50 amino acids, can be synthesized using, for example, the Merrifield solid-phase synthesis method, where amino acids are sequentially added to a growing amino acid chain. See Merrifield, J. Am. Chem. Soc. 85:2149-2146, 1963. Equipment for automated synthesis of polypeptides is commercially available from suppliers such as Perkin Elmer/Applied Biosystems Division, Foster City, Calif.

Polypeptides and fusion polypeptides may also be produced recombinantly by inserting a polynucleotide that encodes the polypeptide into an expression vector and expressing the antigen in an appropriate host. Any of a variety of expression vectors known to those of ordinary skill in the art may be employed. Expression may be achieved in any appropriate host cell that has been transformed or transfected with an expression vector containing a DNA molecule that encodes a recombinant polypeptide. Suitable host cells include prokaryotes, yeast and higher eukaryotic cells. Preferably, the host cells employed are E. coli, mycobacteria, insect, yeast or a mammalian cell line such as COS or CHO. The polynucleotides expressed in this manner may encode naturally occurring antigens, portions of naturally occurring antigens, or other variants thereof.

Expressed polypeptides and fusion polypeptides are generally isolated in substantially pure form. Preferably, the polypeptides and fusion polypeptides are isolated to a purity of at least 80% by weight, more preferably, to a purity of at least 95% by weight, and most preferably to a purity of at least 99% by weight. In general, such purification may be achieved using, for example, the standard techniques of ammonium sulfate fractionation, SDS-PAGE electrophoresis, and affinity chromatography.

The present invention further provides methods for detecting T. cruzi infection in individuals and blood supplies. T. cruzi infection may be detected in any biological sample that contains antibodies. Preferably, the sample is blood, serum, plasma, saliva, cerebrospinal fluid or urine. More preferably, the sample is a blood or serum sample obtained from a patient or a blood supply. Briefly, T. cruzi infection may be detected using any one or more of the polypeptides or fusion polypeptides described above (including portions, epitopes and/or variants thereof), to determine the presence or absence of antibodies to the polypeptide or fusion polypeptide in the sample, relative to a predetermined cut-off value, e.g., determined from uninfected control samples.

There are a variety of assay formats known to those of ordinary skill in the art for using purified antigen to detect antibodies in a sample. See, e.g., Harlow and Lane, Antibodies: A Laboratory Manual, Cold Spring Harbor Laboratory, 1988. In a preferred embodiment, the assay involves the use of polypeptide or fusion polypeptide immobilized on a solid support to bind to and remove the antibody from the sample. The bound antibody may then be detected using a detection reagent that binds to the antibody/fusion polypeptide complex and contains a detectable reporter group. Suitable detection reagents include antibodies that bind to the antibody/fusion polypeptide complex and free polypeptide labeled with a reporter group (e.g., in a semi-competitive assay). Alternatively, a competitive assay may be utilized, in which an antibody that binds to the fusion polypeptide is labeled with a reporter group and allowed to bind to the immobilized antigen after incubation of the antigen with the sample. The extent to which components of the sample inhibit the binding of the labeled antibody to the fusion polypeptide is indicative of the reactivity of the sample with the immobilized fusion polypeptide.

The solid support may be any solid material known to those of ordinary skill in the art to which the fusion polypeptide may be attached. For example, the solid support may be a test well in a microtiter plate, or a nitrocellulose or other suitable membrane. Alternatively, the support may be a bead or disc, formed of glass, fiberglass, latex or a plastic material such as polystyrene or polyvinylchloride. The support may also be a magnetic particle or a fiber optic sensor, such as those disclosed, for example, in U.S. Pat. No. 5,359,681.

The polypeptide or fusion polypeptide may be bound to the solid support using a variety of techniques known to those in the art, which are amply described in the patent and scientific literature. In the context of the present invention, the term “bound” refers to both noncovalent association, such as adsorption, and covalent attachment (which may be a direct linkage between the antigen and functional groups on the support or may be a linkage by way of a cross-linking agent). Binding by adsorption to a well in a microtiter plate or to a membrane is preferred. In such cases, adsorption may be achieved by contacting the polypeptide, in a suitable buffer, with the solid support for a suitable amount of time. The contact time varies with temperature, but is typically between about 1 hour and 1 day. In general, contacting a well of a plastic microtiter plate (such as polystyrene or polyvinylchloride) with an amount of fusion polypeptide ranging from about 10 ng to about 1 μg, and preferably about 100 ng, is sufficient to bind an adequate amount of antigen. Nitrocellulose will bind approximately 100 μg of protein per cm³.

Covalent attachment of the polypeptide or fusion polypeptide to a solid support may generally be achieved by first reacting the support with a bifunctional reagent that will react with both the support and a functional group, such as a hydroxyl or amino group, on the fusion polypeptide. For example, the fusion polypeptide may be bound to supports having an appropriate polymer coating using benzoquinone or by condensation of an aldehyde group on the support with an amine and an active hydrogen on the fusion polypeptide (see, e.g., Pierce Immunotechnology Catalog and Handbook (1991) at A12-A13).

In certain embodiments, the assay is an enzyme linked immunosorbent assay (ELISA). This assay may be performed by first contacting a polypeptide or fusion polypeptide that has been immobilized on a solid support, commonly the well of a microtiter plate, with the sample, such that antibodies to the polypeptide or fusion polypeptide within the sample are allowed to bind to the immobilized polypeptide or fusion polypeptide. Unbound sample is then removed from the immobilized polypeptide or fusion polypeptide and a detection reagent capable of binding to the immobilized antibody-polypeptide complex is added. The amount of detection reagent that remains bound to the solid support is then determined using a method appropriate for the specific detection reagent.

Once the polypeptide or fusion polypeptide is immobilized on the support, the remaining protein binding sites on the support are typically blocked using any suitable blocking agent known to those of ordinary skill in the art, such as bovine serum albumin or Tween 20™ (Sigma Chemical Co., St. Louis, Mo.). The immobilized polypeptide or fusion polypeptide is then incubated with the sample, and antibody (if present in the sample) is allowed to bind to the antigen. The sample may be diluted with a suitable diluent, such as phosphate-buffered saline (PBS) prior to incubation. In general, an appropriate contact time (i.e., incubation time) is that period of time that is sufficient to detect the presence of T. cruzi antibody within a T. cruzi-infected sample. Preferably, the contact time is sufficient to achieve a level of binding that is at least 95% of that achieved at equilibrium between bound and unbound antibody. Those of ordinary skill in the art will recognize that the time necessary to achieve equilibrium may be readily determined by assaying the level of binding that occurs over a period of time. At room temperature, an incubation time of about 30 minutes is generally sufficient.

Unbound sample may then be removed by washing the solid support with an appropriate buffer, such as PBS containing 0.1% Tween 20™. Detection reagent may then be added to the solid support. An appropriate detection reagent is any compound that binds to the immobilized antibody-polypeptide complex and that can be detected by any of a variety of means known to those in the art. Preferably, the detection reagent contains a binding agent (such as, for example, Protein A, Protein G, immunoglobulin, lectin or free antigen) conjugated to a reporter group. Preferred reporter groups include enzymes (such as horseradish peroxidase), substrates, cofactors, inhibitors, dyes, radionuclides, luminescent groups, fluorescent groups and biotin. The conjugation of binding agent to reporter group may be achieved using standard methods known to those of ordinary skill in the art. Common binding agents may also be purchased conjugated to a variety of reporter groups from many sources (e.g., Zymed Laboratories, San Francisco, Calif. and Pierce, Rockford, Ill.).

The detection reagent is then incubated with the immobilized antibody-polypeptide complex for an amount of time sufficient to detect the bound antibody. An appropriate amount of time may generally be determined from the manufacturer's instructions or by assaying the level of binding that occurs over a period of time. Unbound detection reagent is then removed and bound detection reagent is detected using the reporter group. The method employed for detecting the reporter group depends upon the nature of the reporter group. For radioactive groups, scintillation counting or autoradiographic methods are generally appropriate. Spectroscopic methods may be used to detect dyes, luminescent groups and fluorescent groups. Biotin may be detected using avidin, coupled to a different reporter group (commonly a radioactive or fluorescent group or an enzyme). Enzyme reporter groups may generally be detected by the addition of substrate (generally for a specific period of time), followed by spectroscopic or other analysis of the reaction products.

To determine the presence or absence of T. cruzi antibodies in the sample, the signal detected from the reporter group that remains bound to the solid support is generally compared to a signal that corresponds to a predetermined cut-off value. This cut-off value is preferably the average mean signal obtained when the immobilized antigen is incubated with samples from an uninfected patient. In general, a sample generating a signal that is three standard deviations above the mean is considered positive for T. cruzi antibodies and T. cruzi infection. In an alternate preferred embodiment, the cut-off value is determined using a Receiver Operator Curve, according to the method of Sackett et al., Clinical Epidemiology: A Basic Science for Clinical Medicine, p. 106-7 (Little Brown and Co., 1985). Briefly, in this embodiment, the cut-off value may be determined from a plot of pairs of true positive rates (i.e., sensitivity) and false positive rates (100%-specificity) that correspond to each possible cut-off value for the diagnostic test result. The cut-off value on the plot that is the closest to the upper left-hand corner (i.e., the value that encloses the largest area) is the most accurate cut-off value, and a sample generating a signal that is higher than the cut-off value determined by this method may be considered positive. Alternatively, the cut-off value may be shifted to the left along the plot, to minimize the false positive rate, or to the right, to minimize the false negative rate. In general, a sample generating a signal that is higher than the cut-off value determined by this method is considered positive for T. cruzi infection.

In a related embodiment, the assay is performed in a flow-through or strip test format, wherein the polypeptide or fusion polypeptide is immobilized on a membrane such as nitrocellulose. In the flow-through test, antibodies within the sample bind to the immobilized polypeptide or fusion polypeptide as the sample passes through the membrane. A detection reagent (e.g., protein A-colloidal gold) then binds to the antibody-polypeptide complex as the solution containing the detection reagent flows through the membrane. The detection of bound detection reagent may then be performed as described above. In the strip test format, one end of the membrane to which polypeptide or fusion polypeptide is bound is immersed in a solution containing the sample. The sample migrates along the membrane through a region containing detection reagent and to the area of immobilized polypeptide or fusion polypeptide. Concentration of detection reagent at the polypeptide or fusion polypeptide indicates the presence of T. cruzi antibodies in the sample. Such tests can typically be performed with a very small amount (e.g., one drop) of patient serum or blood.

In yet another aspect of this invention, methods are provided for detecting T. cruzi in a biological sample using monospecific antibodies (which may be polyclonal or monoclonal) to one or more T. cruzi polypeptides or fusion polypeptides. Antibodies to purified or synthesized polypeptides may be prepared by any of a variety of techniques known to those of ordinary skill in the art. See, e.g., Harlow and Lane, Antibodies: A Laboratory Manual, Cold Spring Harbor Laboratory, 1988. In one such technique, an immunogen comprising the antigenic polypeptide is initially injected into any of a wide variety of mammals (e.g., mice, rats, rabbits, sheep and goats). In this step, the polypeptides of this invention may serve as the immunogen without modification. Alternatively, particularly for relatively short polypeptides, a superior immune response may be elicited if the polypeptide is joined to a carrier protein, such as bovine serum albumin or keyhole limpet hemocyanin. The immunogen is injected into the animal host, preferably according to a predetermined schedule incorporating one or more booster immunizations, and the animals are bled periodically. Polyclonal antibodies specific for the polypeptide may then be purified from such antisera by, for example, affinity chromatography using the polypeptide coupled to a suitable solid support.

Monoclonal antibodies specific for the antigenic polypeptide of interest may be prepared, for example, using the technique of Kohler and Milstein, Eur. J. Immunol. 6:511-519, 1976, and improvements thereto. Briefly, these methods involve the preparation of immortal cell lines capable of producing antibodies having the desired specificity (i.e., reactivity with the polypeptide of interest). Such cell lines may be produced, for example, from spleen cells obtained from an animal immunized as described above. The spleen cells are then immortalized by, for example, fusion with a myeloma cell fusion partner, preferably one that is syngeneic with the immunized animal. A variety of fusion techniques may be employed. For example, the spleen cells and myeloma cells may be combined with a nonionic detergent for a few minutes and then plated at low density on a selective medium that supports the growth of hybrid cells, but not myeloma cells. A preferred selection technique uses HAT (hypoxanthine, aminopterin, thymidine) selection. After a sufficient time, usually about 1 to 2 weeks, colonies of hybrids are observed. Single colonies are selected and tested for binding activity against the polypeptide. Hybridomas having high reactivity and specificity are preferred.

Monoclonal antibodies may be isolated from the supernatants of growing hybridoma colonies. In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction.

Monospecific antibodies to epitopes of one or more of the polypeptides or fusion polypeptides described herein may be used to detect T. cruzi infection in a biological sample using any of a variety of immunoassays, which may be direct or competitive. Suitable biological samples for use in this aspect of the present invention are as described above. Briefly, in one direct assay format, a monospecific antibody may be immobilized on a solid support (as described above) and contacted with the sample to be tested. After removal of the unbound sample, a second monospecific antibody, which has been labeled with a reporter group, may be added and used to detect bound antigen. In an exemplary competitive assay, the sample may be combined with the monoclonal or polyclonal antibody, which has been labeled with a suitable reporter group. The mixture of sample and antibody may then be combined with polypeptide antigen immobilized on a suitable solid support. Antibody that has not bound to an antigen in the sample is allowed to bind to the immobilized antigen, and the remainder of the sample and antibody is removed. The level of antibody bound to the solid support is inversely related to the level of antigen in the sample. Thus, a lower level of antibody bound to the solid support indicates the presence of T. cruzi in the sample. To determine the presence or absence of T. cruzi infection, the signal detected from the reporter group that remains bound to the solid support is generally compared to a signal that corresponds to a predetermined cut-off value. Such cut-off values may generally be determined as described above. Any of the reporter groups discussed above in the context of ELISAs may be used to label the monospecific antibodies, and binding may be detected by any of a variety of techniques appropriate for the reporter group employed. Other formats for using monospecific antibodies to detect T. cruzi in a sample will be apparent to those of ordinary skill in the art, and the above formats are provided solely for exemplary purposes.

In another aspect of this invention, compositions and methods are provided for the prevention or treatment of T. cruzi infection, and complications thereof, in a mammal. Such compositions generally comprise one or more T. cruzi polypeptides as described herein (including immunogenic portions, epitopes and/or variants thereof) or fusion polypeptides thereof, together with at least one component selected from the group consisting of physiologically acceptable carriers and immunostimulants.

Routes and frequency of administration and polypeptide doses will vary from individual to individual and may parallel those currently being used in immunization against other protozoan infections. In general, the compositions may be administered by injection (e.g., intracutaneous, intramuscular, intravenous or subcutaneous), intranasally (e.g., by aspiration), transdermally, orally or by transcutaneous patch as described, for example, in U.S. Pat. Nos. 5,910,306 and 5,980,898, the disclosures of which are hereby incorporated by reference. Between 1 and 4 doses may be administered for a 2-6 week period. Preferably, two doses are administered, with the second dose 2-4 weeks later than the first. A suitable dose is an amount of polypeptide that is effective to raise antibodies in a treated mammal that are sufficient to protect the mammal from T. cruzi infection for a period of time. In general, the amount of polypeptide present in a dose ranges from about 1 pg to about 100 mg per kg of host, typically from about 10 pg to about 1 mg, and preferably from about 100 pg to about 1 μg. Suitable dose sizes will vary with the size of the animal, but will typically range from about 0.01 mL to about 5 mL for 10-60 kg animal.

While any suitable carrier known to those of ordinary skill in the art may be employed in the compositions of this invention, the type of carrier will vary depending on the mode of administration. For parenteral administration, such as subcutaneous injection, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g., polylactic galactide) may also be employed as carriers for the pharmaceutical compositions of this invention. Suitable biodegradable microspheres are disclosed, for example, in U.S. Pat. Nos. 4,897,268 and 5,075,109.

Any of a variety of immunostimulants may be employed in the compositions of this invention to nonspecifically enhance the immune response. Many adjuvants contain a substance designed to protect the antigen from rapid catabolism, such as aluminum hydroxide or mineral oil, and a nonspecific stimulator of immune responses, such as lipid A, Bortadella pertussis or Mycobacterium tuberculosis. Suitable adjuvants are commercially available as, for example, Freund's Incomplete Adjuvant and Freund's Complete Adjuvant (Difco Laboratories) and Merck Adjuvant 65 (Merck and Company, Inc., Rahway, N.J.). Other suitable adjuvants include alum, biodegradable microspheres, monophosphoryl lipid A and quil A. Other illustrative adjuvants include cytokines, such as GM-CSF or interleukin-2, -7, or -12. Other illustrative adjuvants include Toll-like receptor agonists, such as TLR7 agonists, TLR7/8 agonists, and the like. Still other illustrative adjuvants include imiquimod, gardiquimod, resiquimod, and related compounds.

Certain other illustrative adjuvant systems are designed to induce an immune response predominantly of the Th1 type. High levels of Th1-type cytokines (e.g., IFN-γ, TNF-α., IL-2 and IL-12) tend to favor the induction of cell mediated immune responses to an administered antigen. In contrast, high levels of Th2-type cytokines (e.g., IL-4, IL-5, IL-6 and IL-10) tend to favor the induction of humoral immune responses. Following administration of a vaccine composition, a patient will support an immune response that includes Th1- and Th2-type responses. Within a preferred embodiment, in which a response is predominantly Th1-type, the level of Th1-type cytokines will increase to a greater extent than the level of Th2-type cytokines. The levels of these cytokines may be readily assessed using standard assays. For a review of the families of cytokines, see Mossman & Coffman, Ann. Rev. Immunol. 7:145-173 (1989). Certain illustrative adjuvants for use in eliciting a predominantly Th1-type response include, for example, a combination of monophosphoryl lipid A, preferably 3-de-O-acylated monophosphoryl lipid A (3D-MPL™), together with an aluminum salt (U.S. Pat. Nos. 4,436,727; 4,877,611; 4,866,034; and 4,912,094). CpG-containing oligonucleotides (in which the CpG dinucleotide is unmethylated) also induce a predominantly Th1 response. Such oligonucleotides are well known and are described, for example, in WO 96/02555, WO 99/33488 and U.S. Pat. Nos. 6,008,200 and 5,856,462. Immunostimulatory DNA sequences are also described, for example, by Sato et al., Science 273:352 (1996). Another illustrative adjuvant comprises a saponin, such as Quil A, or derivatives thereof, including QS21 and QS7 (Aquila Biopharmaceuticals Inc., Framingham, Mass.); Escin; Digitonin; or Gypsophila or Chenopodium quinoa saponins. Other illustrative formulations include more than one saponin in the adjuvant combinations of the present invention, for example combinations of at least two of the following group comprising QS21, QS7, Quil A, β-escin, or digitonin.

Yet another illustrative adjuvant system includes the combination of a monophosphoryl lipid A and a saponin derivative, such as the combination of QS21 and 3D-MPL™. adjuvant, as described in WO 94/00153, or a less reactogenic composition where the QS21 is quenched with cholesterol, as described in WO 96/33739. Other formulations comprise an oil-in-water emulsion and tocopherol. Another adjuvant formulation employing QS21, 3D-MPL™ adjuvant and tocopherol in an oil-in-water emulsion is described in WO 95/17210.

Another illustrative adjuvant system involves the combination of a CpG-containing oligonucleotide and a saponin derivative as disclosed in WO 00/09159. Further still, additional illustrative adjuvants include Montanide ISA 720 (Seppic, France), SAF (Chiron, Calif., United States), ISCOMS (CSL), MF-59 (Chiron), the SBAS series of adjuvants (e.g., SBAS-2, AS2′, AS2,″ SBAS-4, or SBAS6, available from SmithKline Beecham, Rixensart, Belgium), Detox, RC-529 (Corixa, Hamilton, Mont.) and other aminoalkyl glucosaminide 4-phosphates (AGPs), such as those described in pending U.S. patent application Ser. Nos. 08/853,826 and 09/074,720, the disclosures of which are incorporated herein by reference in their entireties, and polyoxyethylene ether adjuvants such as those described in WO 99/52549A1.

The following Examples are offered by way of illustration and not by way of limitation.

### EXAMPLE 1

### Sequences Derived from Serological Expression Cloning

### T. cruzi Library Preparation:

A genomic random shear expression library was constructed by sonicating genomic DNA from Trypanosoma cruzi CL strain. Sonication produced fragment sizes of 0.5-2.0 kb. Fifteen micrograms of sonicated DNA was treated with T4 polymerase (NEB) for 15 minutes at 12° C. followed by incubation for 20 minutes at 75° C. to produce blunt ended fragments. EcoRI adaptors were then ligated to the fragments and adaptors were phosphorylated with E. coli polynucleotide kinase. Fragments were next fractionated with a Sephacryl S400 column and finally ligated to a Lambda ZAP Express (Stratagene) vector. Ligated vector was packaged with Gigapack III Gold packaging extract (Stratagene).

### Screening:

The amplified library was plated on LB agarose plates at a concentration of 20,000 plaque forming units (PFU) per 35 plates. After incubation at 42° C. for 4 hrs, nitrocellulose filters soaked in 10 mM IPTG were added and the plates were incubated at 37° C. overnight. Filters were removed and washed 3× with PBS containing 0.1% Tween 20 (PBST), blocked for 1 hr with 1% BSA in PBST, washed 3× with PBST, blocked another 1 hr with 1% Tween 20 in PBS, washed 3× with PBST and then incubated overnight at 4° C. in serum pools from Chagas patients: patient pool #1 (RR mix) and/or pool #2 (Teragenix mix). Both patient serum pools were obtained from RIPA-confirmed low reactive T. cruzi sera. The following day, after washing 3× with PBST, filters were incubated in an alkaline phosphatase secondary antibody goat anti human Ig (IgG, IgA, IgM) for 1 hr at room temperature. Filters were finally washed 3× with PBST, 2× with AP buffer and developed with bromochloroindolyl phosphate/nitroblue tetrazolium (BCIP/NBT) (Invitrogen). Positive clones were purified using the same technique. Phagemid were excised, and resulting plasmid DNA was sequenced and searched against the T. cruzi databases.

### Features and Outcome of Library Screening:

| | |
|---|---|
| Lambda vector: | Lambda Zap Express (Stratagene) |
| Plasmid vector: | pBK-CMV (kanamycin) |
| DNA: | Genomic T. cruzi |
| Library titer: | 2.5 × 10⁸/ml (amplified) (total of 30 ml) |
| Insert size: | 0.5-2.0 Kbp (average = 1.1) |
| Screened: | 20,000 pfu per 35 plates |
| Serum: | Patient pool #1 and pool #2 (from normal donors) |
| | 1:200 dilution |
| Primary Picks: | 31 (Human Ig) from patient pool#1 (from 15 plates) |
| | 47 (Human Ig) from patient pool#2 (from 20 plates) |
| Purified Secondary: | 38 (weak-strong signal) |
| Submitted for | 12 |
| Sequencing: | |

TABLE 1
HITS FROM T. CRUZI GENOMIC LIBRARY SCREENING
WITH POOLED SERUM FROM INFECTED PATIENTS

| | | | | Size | |
|---|---|---|---|---|---|
| Clone | Score | Blastn | Homology | (kDa) | GenBank |
| Tc-2 | 1816 | Tc00.1047053 | dispersed gene family protein | | M90534 T. cruzi |
| | | 509181.9 | (DGF-1 pseudogene) | | protein 1 of DGF-1 |
| Tc-5 | 883 | Tc00.1047053 | hypothetical protein | 14.2 | Novel |
| | | 507757.10 | | | |
| Tc-11 | 3025 | Tc00.1047053 | dispersed gene family protein | | M90534 T. cruzi |
| | | 509181.9 | (DGF-1 pseudogene) | | protein 1 of DGF-1 |
| Tc-12 | 3784 | Tc00.1047053 | dispersed gene family protein | | M90534 T. cruzi |
| | | 509181.9 | (DGF-1 pseudogene) | | protein 1 of DGF-1 |
| Tc-13 | 2765 | Tc00.1047053 | dispersed gene family protein | | M90534 T. cruzi |
| | | 509181.9 | (DGF-1 pseudogene) | | protein 1 of DGF-1 |
| Tc-14 | 2330 | Tc00.1047053 | dispersed gene family protein | | M90534 T. cruzi |
| | | 509659.20 | (DGF-1 pseudogene) | | protein 1 of DGF-1 |
| Tc-15 | 3277 | Tc00.1407053 | Heat shock 70 | 73.2 | X58715 T. cruzi hsp 70 |
| | | 511211.170 | | | mRNA for 70 kDa HSP |
| Tc-19 | | | | | |
| Tc-25 | 2964 | Tc00.1407053 | Heat shock 85 | 81 | M15346 T. cruzi 85 kDa |
| | | 507713.30 | | | heat shock protein |
| Tc-26 | 3175 | Tc00.1407053 | dispersed gene family protein | | |
| | | 510271.20 | (DGF-1 pseudogene) | | |
| Tc-27 | 3362 | Tc00.1407053 | trans-sialidase | | |
| | | 503739.20 | | | |
| Tc-31 | 3276 | | microtubule-associated protein | 125.5 | |

Additional antigenic sequences, referred to as Tc48, Tc60 and Tc70, were also identified. These sequences, plus Tc5, were selected for further analysis. The DNA sequences for Tc5, Tc48, Tc60 and Tc70 are provided in SEQ ID NO: 1, SEQ ID NO: 3, SEQ ID NO: 6 and SEQ ID NO: 8, respectively, with the amino acid sequences for Tc5, Tc60 and Tc70 being provided in SEQ ID NO: 2, SEQ ID NO: 7 and SEQ ID NO: 9. The identified partial amino acid sequence of Tc48 is provided in SEQ ID NO: 4, with the corresponding full-length sequence obtained from the public database being provided in SEQ ID NO: 5.

Given the serological reactivity of the identified T. cruzi antigens, the antigens may be used in the diagnosis and detection of T. cruzi infection in biological samples, particularly in the serological-based detection of T. cruzi infection in patients and/or blood supplies by detecting T. cruzi-reactive antibodies.

From the foregoing, it will be appreciated that, although specific embodiments of the invention have been described herein for the purpose of illustration, various modifications may be made without deviating from the spirit and scope of the invention.

SEQ ID NO: 1-9 are set out in the attached Sequence Listing. The codes for polynucleotide and polypeptide sequences used in the attached Sequence Listing conform to WIPO Standard ST. 25 (1988), Appendix 2.

All references disclosed herein, including patent references and non-patent references, are hereby incorporated by reference in their entirety as if each was incorporated individually.

We claim:

1. An isolated polypeptide comprising an amino acid sequence selected from the group consisting of Tc5 (SEQ ID NO: 2), Tc48 (SEQ ID NO: 5), Tc60 (SEQ ID NO: 7) and Tc70 (SEQ ID NO: 9), or a variant thereof having at least 90% identity thereto.

2. A fusion polypeptide comprising an isolated polypeptide according to claim 1.

3. An isolated polypeptide comprising an amino acid sequence selected from the group consisting of Tc5 (SEQ ID NO: 2), Tc48 (SEQ ID NO: 5), Tc60 (SEQ ID NO: 7) and Tc70 (SEQ ID NO: 9), or an immunogenic portion thereof.

4. A fusion polypeptide comprising an isolated polypeptide according to claim 3.

5. An isolated polynucleotide sequence encoding a polypeptide according to any one of claims 1-4.

6. A recombinant expression vector comprising a polynucleotide sequence according to claim 5.

7. A host cell transformed or transfected with an expression vector according to claim 6.

8. A method for detecting T. cruzi infection in a biological sample, comprising:
(a) contacting the biological sample with a polypeptide of any one of claims 1-4; and
(b) detecting in the biological sample the presence of antibodies that bind to the polypeptide, and thereby detecting T. cruzi infection in the biological sample.

9. The method of claim 8, wherein the biological sample is selected from the group consisting of blood, serum, plasma, saliva, cerebrospinal fluid and urine.

10. A diagnostic kit for detecting T. cruzi infection in a biological sample, comprising:
(a) a polypeptide of any one of claims 1-4; and
(b) a detection reagent.

11. The kit of claim 10, wherein the detection reagent comprises a reporter group.

12. The kit of claim 10, wherein the reporter group is selected from the group consisting of: enzymes, substrates, cofactors, inhibitors, dyes, radionuclides, luminescent groups, fluorescent groups, and biotin.

13. A composition comprising a polypeptide according to any one of claims 1-4 and at least one component selected from the group consisting of physiologically acceptable carriers and immunostimulants.

14. A method for the prevention or treatment of T. cruzi infection comprising administering to a patient a composition of claim 13.


SEQUENCE LISTING (9 sequences)

SEQ ID NO: 1 - DNA, 447 bp, Trypanosoma cruzi
atgggcagcagccatcatcatcatcatcacagcagcggcctggtgccgcgcggcagccatatggccgcaggcggagagctggcggcaacagcagctgcagtgcggggacagtttgcatcgtgtgcccgtgggggaaggacgacatggcgcaggatgggcttctggaggctgagcttttcaccccggtggatgagagcctctagtccagcagacgtgctgagcatggggagaaggagctgtgaccgccccgtattttggcctcagagccagagtgcaccctgtcgaaggcaaaaaggggcgaagtcgacacaatgcgacttgtccacgaagcgcttcttcctcccatgctctggcagaggcgtgccgggaggatactgcgaacagttgcgcttttgttgttggctgtggtggcggggagggcggaggggcagactcaactgaaagctt

SEQ ID NO: 2 - protein, 146 aa, Trypanosoma cruzi
MGSSHHHHHHSSGLVPRGSHMAAGGELAATAAAVRGQFASCARGGRTTWRRMGFWRLSFSPRWMRASSPADVLSMGRRSCDRPVFWPQSQSAPCRRQKGAKSTQCDLSTKRFFLPCSGRGVPGGYCEQLRFCCWLWWRGGRRGRLN

SEQ ID NO: 3 - DNA, 383 bp, Trypanosoma cruzi
atgggcagcagccatcatcatcatcatcacagcagcggcctggtgccgcgcggcagccatatgcccaaaaagaccggtggcaaaaagaaggggcaaagttctccggatggctctgagccgcggaaacgaaagaacaacaaaaaggcgacaatggagccgcgggacgtggatgagatgcagaagctgcaggaacttttaggggacgaggaacagccgttgggtgtctccaagaaatcgctagagggcttattgtcccttcggcagccgcaggagttggcggtgaggcttgcgcaatctctctcctccctgcgcgcgcggcttgcggagttggagttggagaggcttaaccgtgggagcgaggcgccggggctgtcgaacatcgt

SEQ ID NO: 4 - protein, 127 aa, Trypanosoma cruzi
MGSSHHHHHHSSGLVPRGSHMPKKTGGKKKGQSSPDGSEPRKRKNNKKATMEPRDVDEMQKLQELLGDEEQPLGVSKKSLEGLLSLRQPQELAVRLAQSLSSLRARLAELELERLNRGSEAPGLSNI

SEQ ID NO: 5 - protein, 400 aa, Trypanosoma cruzi
MPKKTGGKKKGQSSPDGSEPRKRKNNKKATMEPRDVDEMQKLQELLGDEEQPLGVSKKSLEGLLSLRQPQELAVRLAQSLSSLRARLAELELERLNRGSEAPGLSNIVARRAQEKAEKLELEIQKTERVTRRLKIISSLVGHIIRLREKTLTETHTAMEAEVQSLQEKIRVNEELIRERFVSRVNMLHRYWLWRTLQELGDQTVGWTFEEELARGPRYRTLGVQNNIVSETLEQQLSWLLVFAEKEKIFREHVRRLELLVEELTDINDALEEALTCRVCGLLFEDPVLFWPCGHVFCLVCFDTLSIAPSLFRCPTCGSMGSEGYVHNLLIAESVAKWMFKDAGYGDIHGALSLIRLHLSKFRKEVISSRVAQLHQQLTEARQKETKVEELSQMDITYRDF

SEQ ID NO: 6 - DNA, 515 bp, Trypanosoma cruzi
ccatgggcagcagccatcatcatcatcatcacagcagcggcctggtgccgcgcggcagccatatgtgcattgctcttggcatcgtcgcggaggatgatgaggcatggcgaattgccagcgaggctgtcgctgcagaaaaggagcctgtcttcagcgggaacaacggcccttttgtagatgtctggtttggcgaacagaaactctttggcctcgttcaacgcgttgctccaaacgactttattcaggtcgcccaggagtgtggcgagaagagcgatgacgccgcagcgacgttgcggatgcgtgtgacgcacaacgtctcttttgtccttcacctctcgtcggtgccgcatgcgatgctgcaggcacggggagcgcccgaggacaagtttgtgaacttcatgcaacttgtcgtggattacgcttcgctgctgcggcgcgggatgaaggatgagtttcttggcgtcgatcccgagtccgatgcggagtacatacgcttcacgccccagtgaaagctt

SEQ ID NO: 7 - protein, 168 aa, Trypanosoma cruzi
MGSSHHHHHHSSGLVPRGSHMCIALGIVAEDDEAWRIASEAVAAEKEPVFSGNNGPFVDVWFGEQKLFGLVQRVAPNDFIQVAQECGEKSDDAAATLRMRVTHNVSFVLHLSSVPHAMLQARGAPEDKFVNFMQLVVDYASLLRRGMKDEFLGVDPESDAEYIRFTPQ

SEQ ID NO: 8 - DNA, 526 bp, Trypanosoma cruzi
ccatgggcagcagccatcatcatcatcatcacagcagcggcctggtgccgcgcggcagccatatgtgttcccaatacggacccttgaggaactcatcgcagcgtggaagaccgatcgctgtgtctgttgccaactcgtgtcttcgcgtagcggccacgcagaaagtttctaagccaccttctacagttcacccccggaatattggccggcaagcgactgaggactcgatgaccaatgaactcaaaggccttgctggagtctaccagcaccaacggagcccgatggggtctgcagtggagctggcttccaacaccgctcttcctgggaaggttcacttggaattaatcgtttctgttatgctcaaattcgtttatcaggtgtgccagctgcatcgtcgtggtacgcatactacggcacgcatactggtgcggacgaggcgtccacagccaaagcagtctcctcaatgccattttctcagcaaccttacccacgatggaaggacagctgattggtcgatatgaagctt

SEQ ID NO: 9 - protein, 172 aa, Trypanosoma cruzi
MGSSHHHHHHSSGLVPRGSHMCSQYGPLRNSSQRGRPIAVSVANSCLRVAATQKVSKPPSTVHPRNIGRQATEDSMTNELKGLAGVYQHQRSPMGSAVELASNTALPGKVHLELIVSVMLKFVYQVCQLHRRGTHTTARILVRTRRPQPKQSPQCHFLSNLTHDGRTADWSI